(19) United States
(12) Patent Application Publication
(10) Pub. No.: US 2008/0269665 A1
(43) Pub. Date: Oct. 30, 2008

(54) CHEMOTHERAPY DELIVERY DEVICE

(76) Inventor: Johan Petersen, Streamwood, IL (US)

Correspondence Address: TREXLER, BUSHNELL, GIANGIORGI,;BLACKSTONE & MARR, LTD., 105 WEST ADAMS STREET, SUITE 3600, CHICAGO, IL, 60603, US

(21) Appl. No.: 11/739,341

(22) Filed: Apr. 24, 2007

(51) Int. Cl. A61N 1/30

(52) U.S. Cl. 604/21

(57) ABSTRACT

A system for delivering medicament to a patient is described. In a first embodiment, the system has a positive ion generator having an input and an output, a device for supplying a medicament, the device in fluid communication with the input, and a nebulizing catheter in fluid communication with the output, the nebulizing catheter configured to receive medicament ionized by the positive ion generator and to deliver the medicament to the patient. In another embodiment, the system has an intracorporeal catheter in fluid communication with the output, the intracorporeal catheter configured to receive medicament ionized by the positive ion generator and to deliver the medicament to the patient. In yet another embodiment, the system has a nebulizing catheter having an input and an output, a device for supplying a medicament, the device in fluid communication with the input, and a positive ion generator in fluid communication with the output, the positive ion generator configured to receive medicament nebulized by the by the nebulizing catheter and to deliver the medicament to the patient.

In another embodiment, the invention is a method of delivering a medicament to a patient, with the steps of ionizing the medicament with a positive ion generator and delivering the ionized medicament to the patient with a nebulizing catheter. In yet another embodiment, the steps are ionizing a medicament with a positive ion generator and delivering the ionized medicament to the patient with an intracorporeal catheter. In yet another embodiment, the steps are nebulizing a medicament, ionizing the nebulized medicament with a positive ion generator, and delivering the nebulized and ionized medicament to the patient.

### BACKGROUND OF THE INVENTION

This invention relates to the art of delivering medicaments to the body of an animal. More particularly, the invention relates to delivering medicaments such as chemotherapy agents to internal organs of animals, particularly humans.

Various medicaments must be delivered internally to a patient by means other than oral. Some therapeutic agents are delivered intravenously, through an intracorporeal catheter inserted into the patient's blood supply. Other therapeutic agents are delivered in an aerosol or nebula form into the lungs. Additionally, some therapeutic agents are delivered in a nebulized form directly to internal body organs, by use of an insufflator. For example, in one system an insufflator pumps an inert gas, such as carbon dioxide, into the peritoneal cavity to distend the abdomen, and a nebulizing catheter supplies the therapeutic agent directly to the surface of the internal body organs. This process is described in, for example, United States Published Patent Application No. 2005/0137529 A1, System and Method for Delivering a Substance to a Body Cavity, the disclosure of which is incorporated herein by reference.

Problems arise in these methods of delivering medicaments. Some therapeutic agents, such as chemotherapy drugs and pain-relief medication, are needed at specific body sites and not needed elsewhere. A therapeutic agent injected into the blood supply will be delivered to all body organs. Similarly, a nebulized medicament will be delivered to all surfaces of the lung or all surfaces of the insufflated peritoneal cavity. These processes waste those portions of the medicament delivered to body organs where the medicament is not needed. Additionally, some medicaments, in particular chemotherapeutic agents, are toxic and cause problems in non-malignant portions of the patient's body.

Malignant cells are naturally negatively-charged, due to the excess of nucleophilic DNA. Some chemotherapeutic agents take advantage of this property by binding to nucleophilic sites, such as on guanine in DNA.

If the chemotherapeutic agent were positively charged, that agent would be drawn to negatively-charged sites such as cancerous tumors, thereby concentrating the delivery of the agent to the site where the agent is most needed. Accordingly, a need exists for a medicament-delivery system that will increase the positive electrical potential of the medicament. The present invention meets this need.

### BRIEF SUMMARY OF THE PREFERRED EMBODIMENTS OF THE INVENTION

The present invention is a system and a method of delivering a medicament to the body of a patient, human or otherwise. The system of the present invention, in a first embodiment, is a positive ion generator having an input and an output, a device for supplying a medicament, the device in fluid communication with the input, and a nebulizing catheter in fluid communication with the output, the nebulizing catheter configured to receive medicament ionized by the positive ion generator and to deliver the medicament to the patient. In another embodiment, the system is an intracorporeal catheter in fluid communication with the output, the intracorporeal catheter configured to receive medicament ionized by the positive ion generator and to deliver the medicament to the patient. In yet another embodiment, the system is a nebulizing catheter having an input and an output, a device for supplying a medicament, the device in fluid communication with the input, and a positive ion generator in fluid communication with the output, the positive ion generator configured to receive medicament nebulized by the by the nebulizing catheter and to deliver the medicament to the patient.

The method of the present invention, in another embodiment, is the steps of ionizing the medicament with a positive ion generator and delivering the ionized medicament to the patient with a nebulizing catheter. In yet another embodiment, the steps are ionizing a medicament with a positive ion generator and delivering the ionized medicament to the patient with an intracorporeal catheter. In yet another embodiment, the steps are nebulizing a medicament, ionizing the nebulized medicament with a positive ion generator, and delivering the nebulized and ionized medicament to the patient.

### BRIEF DESCRIPTION OF THE SEVERAL VIEWS OF THE DRAWINGS

The organization and manner of the structure and operation of the invention, together with further objects and advantages thereof, may best be understood by reference to the following description, taken in connection with the accompanying non-scale drawings, wherein like reference numerals identify like elements in which:

FIG. 1A is a diagram of the system of the preferred embodiment of the invention;

FIG. 1B is a diagram of the system of another embodiment of the invention;

FIG. 2 is a flow chart of the method of the preferred embodiment of the invention;

FIG. 3 is a diagram of the system of another embodiment of the invention;

FIG. 3A is a block diagram of the method of another embodiment of the invention;

FIG. 4A is a diagram of another embodiment of the invention;

FIG. 4B is a diagram of another embodiment of the invention; and

FIG. 5 is a flow chart of the method of another embodiment of the invention.

### DETAILED DESCRIPTION OF THE PREFERRED EMBODIMENTS OF THE INVENTION

While the invention may be susceptible to embodiment in different forms, there is shown in the drawings, and herein will be described in detail, specific embodiments with the understanding that the present disclosure is to be considered an exemplification of the principles of the invention, and is not intended to limit the invention to that as illustrated and described herein. The embodiments described herein apply to use on a human, but can be used on any animal.

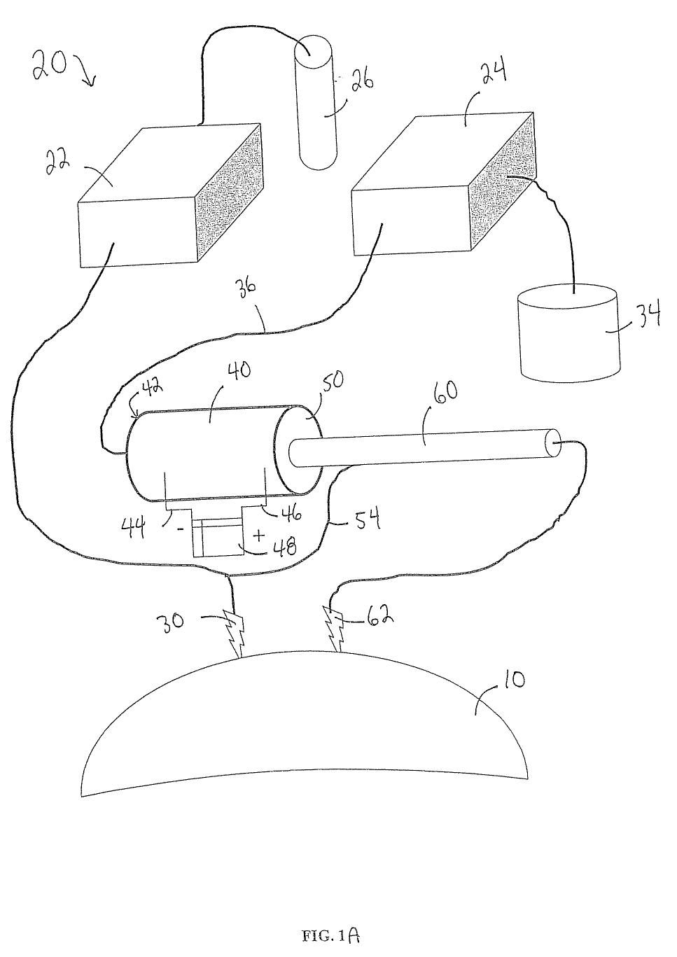

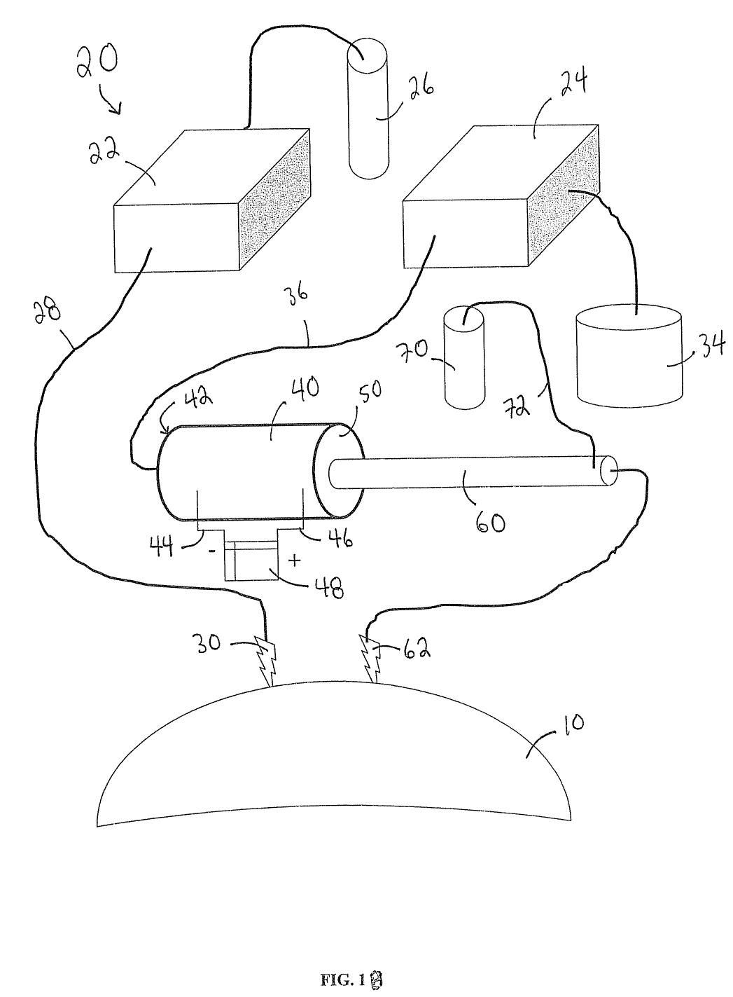

In a first embodiment, the present invention is a chemotherapy delivery system 20 for delivery of a nebulized chemotherapeutic agent to the patient's lungs or peritoneal cavity, as shown in FIG. 1A. System 20 has an insufflator 22 and a medicament-supply device 24. In the preferred embodiment, the medicament-supply device is a pump, but other means of supplying medicament can be used, such as gravity feed or a syringe. System 20 also has conventional control valves, pressure gauges, flow gauges, and pressure-relief systems. The valves, gauges, pressure-relief systems, and tubing and delivery lines of system 20 are preferably made of medical-grade materials.

Insufflator 22 is preferably a conventional laparoscopic insufflator, such as is described in U.S. Pat. No. 6,299,592, Laparoscopic Insufflator, the disclosure of which is incorporated herein by reference. Insufflator 22 is connected to a supply of gas 26, which is delivered to a patient's peritoneal cavity 10 via insufflation gas line 28 to trocar 30. In a first embodiment, gas 26 is carbon dioxide. In another embodiment, gas 26 is an inert gas such as helium or argon. In yet other embodiments, other gases such as nitrous oxide are used. Mixtures of gases can be used as well, such as, for example, a mixture of oxygen and helium. Because insufflator 22 provides gas 26 under pressure to peritoneal cavity 10, trocar 30 should be understood to incorporate a pressure relief valve or other means as known in the art to prevent peritoneal cavity 10 from becoming overpressurized.

Device 24 is connected to a supply of a medicament 34. Medicament 34 in the preferred embodiment is a chemotherapeutic agent on which a positive charge is desired to form an attraction to negatively-charged tumors. In other embodiments, other medicaments can be used, including other chemotherapeutic agents, pain relief medicaments, antibiotics, tissue adhesion agents, or any other therapeutic agents on which a positive charge is desired.

Device 24 supplies medicament 26 through delivery line 36 to generating section 40. Device 24 is a conventional pump configured to deliver a controlled supply of a medicament. Pump 24 can be integral to insufflator 22 or can be a separate unit.

Generating section 40 is a positive ion generator. Preferably, generating section 40 is a tubular member having a fluid pathway therethrough. Within generating section 40 is an induction electrode 44 and a discharge electrode 46 coupled to a voltage source 48. In the preferred embodiment, voltage source 48 is the same power supply as is used for insufflator 22. In other embodiments, voltage source 48 is the same power supply as is used for device 24. In yet another embodiment, voltage source 48 is a separate power supply, such as a battery.

The downstream end 50 of generating section 40 is in fluid communication with nebulizing catheter 60. Nebulizing catheter 60 is a fluid delivery device for delivering positively-charged medicament 34 to the patient's peritoneal cavity 10 or other location within the patient through trocar 62, and is preferably as described in U.S. Pat. No. 5,694,223, Nebulizing Catheter System and Methods of Manufacture, the disclosure of which is incorporated herein by reference. Nebulizing catheter also receives nebulizing gas 26 from insufflator 22 through delivery line 54.

In a second embodiment, as shown in FIG. 1B, nebulizing catheter 60 has a separate gas supply 70. Gas supply 70 conveys gas through delivery line 72 to nebulizing catheter 60. Gas supply 70 is preferably bottled, high-pressure compressed gas, such as carbon dioxide, an inert gas such as helium or argon, nitrous oxide, or some other suitable gas. In another embodiment, gas supply 70 is a mixture of gases, such as, for example, a mixture of oxygen and helium. Delivery line 54 is not needed in this embodiment. The remaining components of system 20 in FIG. 1B are the same as previously described for FIG. 1A.

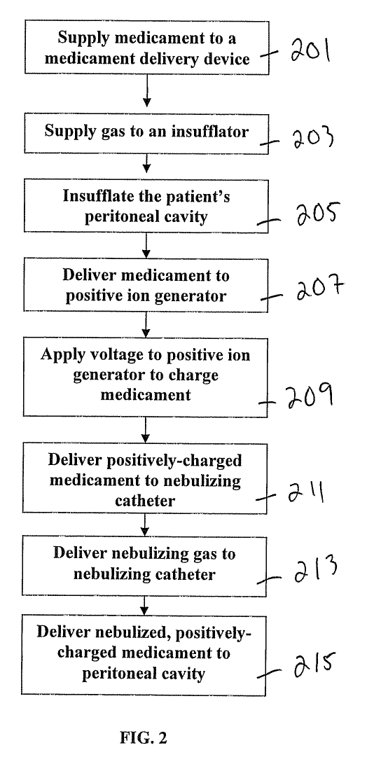

The use of system 20 is shown in flow-chart form in FIG. 2. Medicament 34 is supplied to device 24 (step 201) and gas 26 is supplied to insufflator 22 (step 203). Insufflation gas 26 flows through gas line 28 to trocar 30, whereupon gas 26 inflates the patient's peritoneal cavity 10 in a conventional manner (step 205). Device 24 delivers medicament 34 through delivery line 36 to upstream end 42 of generating section 40 (step 207).

A voltage is applied across electrodes 44, 46, which applies a positive charge to medicament 34 as it flows through generating section 40 (step 209). The positively-charged medicament 34 exits downstream end 50 and enters nebulizing catheter 60 (step 211). Nebulizing gas 26 also enters nebulizing catheter 60.

In a first embodiment, nebulizing gas 26 enters nebulizing catheter 60 from delivery line 54 (step 213). In another embodiment, nebulizing gas 70 enters nebulizing catheter 60 from a compressed gas source (step 213). Nebulizing catheter 60 then nebulizes medicament 34 with gas 26 or gas 70 and delivers nebulized, positively-charged medicament 34 into the patient's body cavity (step 215).

In other embodiments, nebulized, positively-charged medicament 34 is introduced through nebulizing catheter 60 directly into the lungs, another body cavity, a surgical incision, or other location of the patient, rather than into the insufflated peritoneal cavity 10. In these embodiments, trocar 62 is not needed.

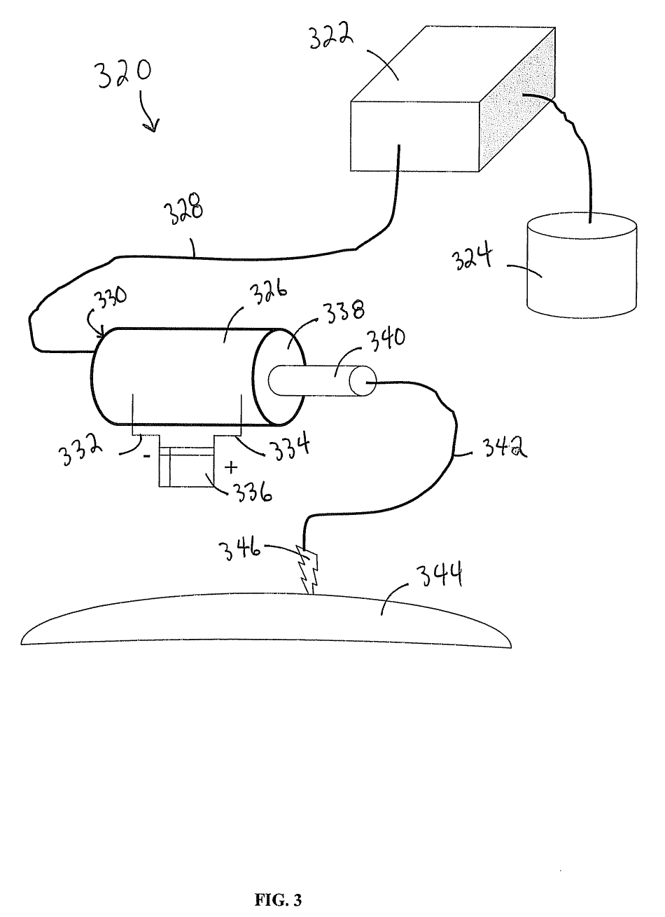

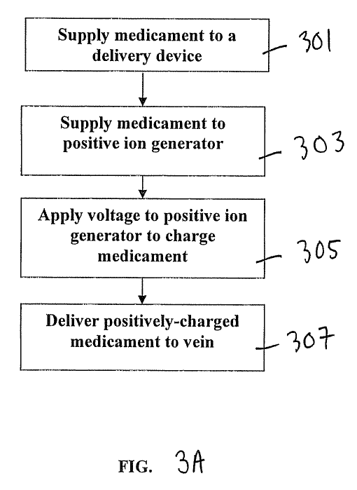

In yet another embodiment, the present invention is a chemotherapy delivery system 320 for delivery of a chemotherapeutic agent to a patient's blood supply, as shown in FIG. 3. System 320 has a medicament-supply device 322. In the preferred embodiment, the medicament-supply device 322 is a pump, but other means of supplying medicament can be used, such as gravity feed or a syringe. Device 322 is preferably a conventional pump configured to deliver a controlled supply of a medicament 324 in a manner known in the art.

Device 322 is connected to a supply of medicament 324. In the preferred embodiment, medicament 324 is a chemotherapeutic agent, such as cisplatin (cis-diamminedichloroplatinum(II), or PT(NH₃)₂Cl₂). In other embodiments, other chemotherapeutic medicaments are used.

Device 322 is in fluid communication with generating section 326, through delivery line 328, which enters the upstream end 330 of generating section 326. Generating section 326 is a positive ion generator. Generating section 326 is preferably a tubular member having a fluid pathway therethrough. Within the fluid pathway are an induction electrode 332 and a discharge electrode 334 coupled to a voltage source 336. In the preferred embodiment, voltage source 336 is the same power supply as is used for device 322. In another embodiment, voltage source 336 a separate power supply.

The downstream end 338 of generating section 326 connects to delivery line 340, which is removably connected to intracorporeal catheter 342. Catheter 342 is preferably a peripheral intravenous line insertable into a vein 344 of a patient through a cannula 346. In other embodiments, intracorporeal catheter 342 is a central intravenous line, a peripherally inserted central catheter, a central venous line, or an arterial line. In yet another embodiment, intracorporeal catheter 342 couples to implanted port in the patient.

System 320 also has conventional control valves, pressure gauges, flow gauges, and pressure-relief systems. The valves, gauges, pressure-relief systems, and tubing and delivery lines of system 320 are preferably made of medical-grade materials known in the art.

The use of system 320 is shown in flow-chart form in FIG. 3A. To use system 320, medicament 324 is supplied to device 322 (step 301).

Device 322 delivers medicament 324 through delivery line 328 into the upstream end 330 of generating section 326 (step 303). A voltage is applied across electrodes 332, 334, to apply a positive charge to medicament 324 as it flows through the fluid pathway of generating section 326. Medicament 324 therefore becomes positively charged from the generation of positive ions (step 305). Positively-charged medicament 324 exits generating section 326 at its downstream end 338, and flows through delivery line 340 to catheter 342, whereupon it enters the patient in a conventional manner (step 307).

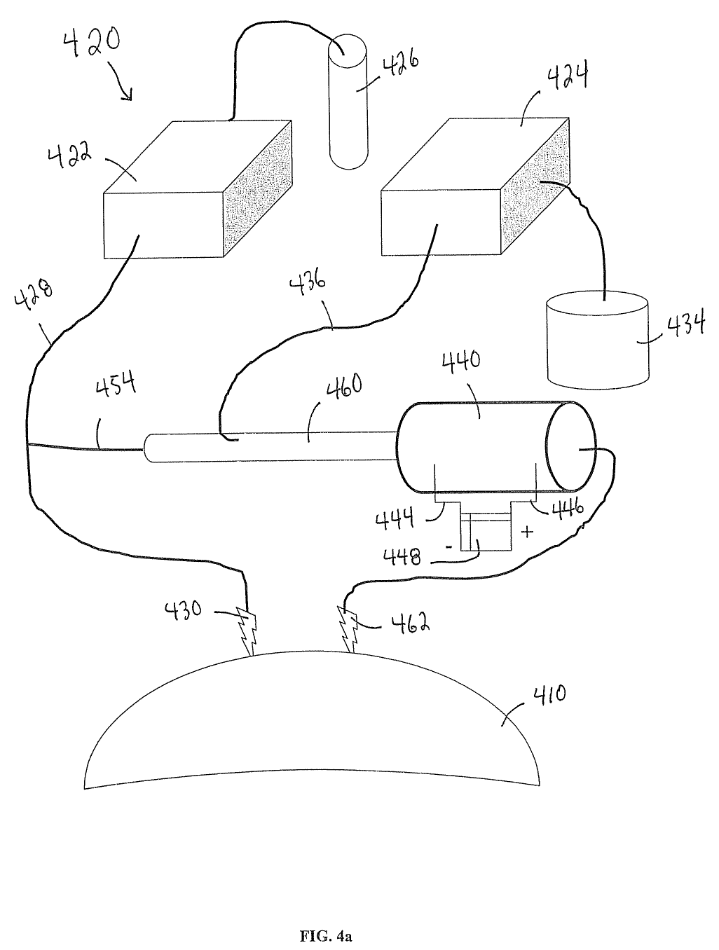

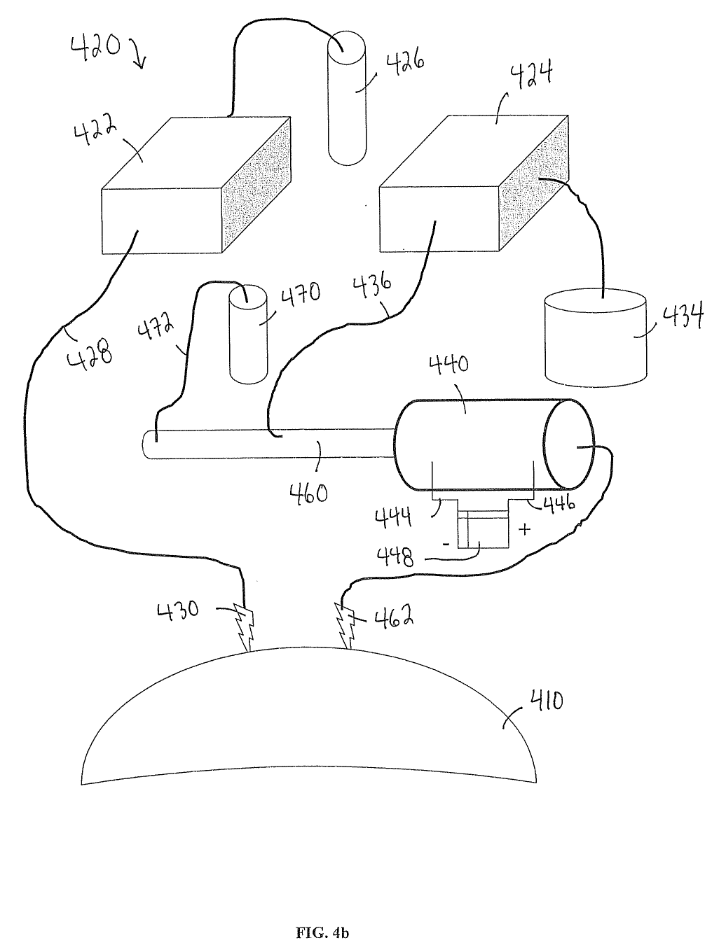

In yet another embodiment, the medicament is nebulized before being ionized, as shown in FIGS. 4A and 4B. In a first embodiment, the present invention is a delivery system 420 for delivery of a positively-charged agent to a patient, as shown in FIG. 4A. System 420 has an insufflator 422 and a medicament-supply device 424.

In the preferred embodiment, medicament-supply device 424 is a conventional pump configured to deliver a controlled supply of a medicament in a manner known in the art. In other embodiments, medicament-supply device 424 is another means of supplying medicament, such as gravity feed or a syringe. Medicament-supply device 424 can be a separate unit, as illustrated, or can be integral to insufflator 22.

System 420 also has conventional control valves, pressure gauges, flow gauges, and pressure-relief systems. The valves, gauges, pressure-relief systems, and tubing and delivery lines of system 420 are preferably made of medical-grade materials known in the art.

Insufflator 422 is preferably a conventional laparoscopic insufflator, such as is described in U.S. Pat. No. 6,299,592, Laparoscopic Insufflator, the disclosure of which is incorporated herein by reference. Insufflator 422 is connected to a supply of gas 426, which is delivered to a patient's peritoneal cavity 410 via insufflation gas line 428 to trocar 430. In a first embodiment, gas 426 is carbon dioxide. In another embodiment, gas 426 is an inert gas such as helium or argon. In yet other embodiments, other gases such as nitrous oxide are used. Mixtures of gases can be used as well, such as, for example, a mixture of oxygen and helium. Because insufflator 422 provides gas 426 under pressure to peritoneal cavity 410, trocar 430 should be understood to incorporate a pressure relief valve or other means as known in the art to prevent peritoneal cavity 410 from being overpressurized.

Medicament-supply device 424 is connected to a supply of a medicament 434. Medicament 434 in the preferred embodiment is a chemotherapeutic agent on which a positive charge is desired to form an attraction to negatively-charged tumors. In other embodiments, other medicaments can be used, including other chemotherapeutic agents, pain relief medicaments, antibiotics, tissue adhesion agents, or any other therapeutic agents on which a positive charge is desired.

Medicament-supply device 424 supplies medicament 426 through delivery line 436 to nebulizing catheter 460. Nebulizing catheter 460 is a fluid delivery device for delivering medicament 434 to the patient's peritoneal cavity 410 through trocar 462, or to another location within the patient, and is preferably as described in U.S. Pat. No. 5,694,223, Nebulizing Catheter System and Methods of Manufacture, the disclosure of which is incorporated herein by reference. Nebulizing catheter 460 in this embodiment receives nebulizing gas 426 from insufflator 422 through delivery lines 428 and 454.

Nebulizing catheter 460 is in fluid communication with generating section 440. Generating section 40 is a positive ion generator. Preferably, generating section 440 is a tubular member having a fluid pathway therethrough. Within the fluid pathway are an induction electrode 444 and a discharge electrode 446 coupled to a voltage source 448. In the preferred embodiment, voltage source 448 is the same power supply as is used for insufflator 422. For example, wires can run within the tubular wall of delivery lines 428 and 454 to delivery voltage to generating section 440, which in this embodiment is at the tip of nebulizing catheter 460. In other embodiments, voltage source 448 is the same power supply as is used for pump 424. In yet another embodiment, voltage source 448 is a separate power supply.

Generating section 440 is in fluid communication with trocar 462, which is inserted into the peritoneal cavity 410 of a patient.

In a second embodiment, as shown in FIG. 4B, nebulizing catheter 460 has a separate gas supply 470. Gas supply 470 is preferably bottled, high-pressure compressed gas, such as carbon dioxide, an inert gas such as helium or argon, nitrous oxide, or some other suitable gas. In another embodiment, gas supply 470 is a mixture of gases, such as, for example, a mixture of oxygen and helium. Gas 470 flows through delivery line 472 to nebulizing catheter 460. Delivery line 454 is accordingly not needed in this embodiment. The other components of system 420 in FIG. 4B are the same as previously described for FIG. 4A.

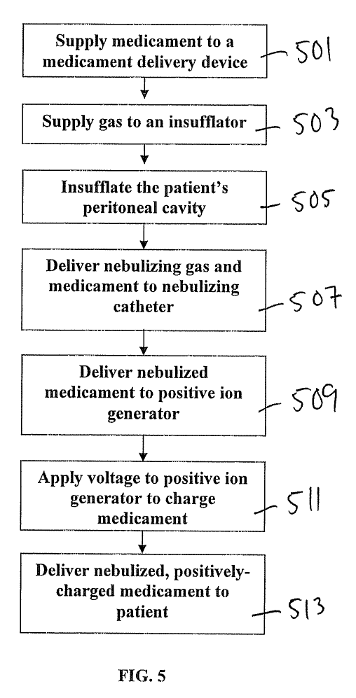

The use of system 420 is shown in flow-chart form in FIG. 5. Medicament 434 is supplied to device 424 (step 501) and gas 426 is supplied to insufflator 422 (step 503). Insufflation gas 426 flows through gas line 428 to trocar 430, whereupon gas 426 inflates the patient's peritoneal cavity 410 in a conventional manner (step 505). Device 424 pumps medicament 434 through delivery line 436 to nebulizing catheter 460 while gas 426 is also delivered to nebulizing catheter 460 (step 507).

In one embodiment, gas 426 nebulizing catheter 60 from delivery line 454. In another embodiment, nebulizing gas 470 enters nebulizing catheter 460 from gas supply 470. Nebulizing catheter 460 then nebulizes medicament 434 with gas 426 or gas 470 and delivers nebulized medicament 434 to the upstream end 442 of generating section 440 (step 509).

A voltage is applied across electrodes 444, 46, thereby applying a positive charge to medicament 434 as it flows through generating section 440 (step 511). The positively-charged, nebulized medicament 434 exits downstream end 450 into the patient's body cavity (step 513).

In another embodiment, nebulized, positively-charged medicament 34 is introduced directly into the lungs, another body cavity, a surgical incision, or other location of the patient, rather than into the insufflated peritoneal cavity 410. In this embodiment, trocar 462 is not needed.

The invention is described for use with a chemotherapeutic agent, to form an attraction to negatively-charged tumors. In other embodiments, the system and method of the present invention are used with any pain relief medicaments, antibiotics, tissue adhesion agents, or other therapeutic agents on which a positive charge is desired.

While preferred embodiments of the present invention are shown and described, it is envisioned that those skilled in the art may devise various modifications of the present invention without departing from the spirit and scope of the appended claims.

1. A system for delivering a medicament to a patient, said system comprising:
a positive ion generator having an input and an output;
a device for supplying the medicament, said device in fluid communication with said input; and
a nebulizing catheter in fluid communication with said output, said nebulizing catheter configured to receive the medicament ionized by said positive ion generator and to deliver the medicament to the patient.

2. The system of claim 1, wherein said positive ion generator comprises an induction electrode and a discharge electrode coupled to a voltage source.

3. The system of claim 1, further comprising an insufflator in fluid communication with said input of said positive ion generator.

4. The system of claim 1, further comprising an insufflator in fluid communication with the patient.

5. The system of claim 1, wherein said device for supplying medicament comprises at least one of a pump, a gravity feed, and a syringe.

6. An system for delivering a fluid medicament to a patient, said system comprising:
a positive ion generator having an input and an output;
a device for supplying the medicament, said device in fluid communication with said input; and
an intracorporeal catheter in fluid communication with said output, said intracorporeal catheter configured to receive the medicament ionized by said positive ion generator and to deliver the ionized medicament to a blood supply of the patient, said intracorporeal catheter comprising at least one of a peripheral intravenous line, a central intravenous line, a peripherally inserted central catheter, a central venous line, and an arterial line.

7. The system of claim 6, wherein said positive ion generator comprises an induction electrode and a discharge electrode coupled to a voltage source.

8. The system of claim 6, wherein said device for supplying medicament comprises at least one of a pump, a gravity feed, and a syringe.

9. An system for delivering a medicament to a patient, said system comprising:
a nebulizing catheter having an input and an output;
a device for supplying the medicament, said device in fluid communication with said input; and
a positive ion generator in fluid communication with said output, said positive ion generator configured to receive the medicament nebulized by said by said nebulizing catheter and to deliver the ionized medicament to the patient.

10. The system of claim 9, wherein said positive ion generator comprises an induction electrode and a discharge electrode coupled to a voltage source.

11. The system of claim 9, further comprising an insufflator in fluid communication with said input of said nebulizing catheter.

12. The system of claim 9, further comprising an insufflator in fluid communication with the patient.

13. The system of claim 9, wherein said device for supplying medicament comprises at least one of a pump, a gravity feed, and a syringe.

14. A method for delivering a medicament to a patient, said method comprising:
ionizing the medicament with a positive ion generator; and
delivering the ionized medicament to the patient with a nebulizing catheter.

15. The method of claim 14, further comprising insufflating the patient.

16. A method for delivering a medicament to a patient, said method comprising:
ionizing the medicament with a positive ion generator;
delivering the ionized medicament to the patient with an intracorporeal catheter.

17. A method for delivering a medicament to a patient, said method comprising:
nebulizing the medicament;
ionizing the nebulized medicament with a positive ion generator;
delivering the nebulized and ionized medicament to the patient.

18. The method of claim 17, further comprising insufflating the patient.

19. A system for delivering a fluid medicament to a blood supply of a patient, said system comprising:
a positive ion generator having an upstream end and a downstream end;
a medicament-supply device connected to said upstream end; and
an intracorporeal catheter connected to said downstream end, said intracorporeal catheter comprising at least one of a peripheral intravenous line, a central intravenous line, a peripherally inserted central catheter, a central venous line, and an arterial line.

20. The system of claim 19, wherein said positive ion generator comprises an induction electrode and a discharge electrode coupled to a voltage source.

21. The system of claim 19, wherein said medicament supply device comprises at least one of a pump, a gravity feed, and a syringe.

22. (canceled)

23. (canceled)

24. The system of claim 19, wherein said intracorporeal catheter couples to an implanted port.

25. (canceled)

26. The system of claim 6, wherein said intracorporeal catheter couples to an implanted port.

27. A system for delivering a fluid medicament to a patient, said system comprising:
a positive ion generator having an upstream end and a downstream end, said positive ion generator comprising an induction electrode and a discharge electrode coupled to a voltage source, said induction electrode and said discharge electrode contained within said positive ion generator;
a medicament-supply device connected to said upstream end; and
an intracorporeal catheter connected to said downstream end, said intracorporeal catheter comprising at least one of a peripheral intravenous line, a central intravenous line, a peripherally inserted central catheter, a central venous line, and an arterial line.

28. The system of claim 27, wherein said medicament supply device comprises at least one of a pump, a gravity feed, and a syringe.

29. The system of claim 27, wherein said intracorporeal catheter couples to an implanted port.
